(19) United States
(12) Patent Application Publication
(10) Pub. No.: US 2001/0031939 A1
(43) Pub. Date: Oct. 18, 2001

(54) Arm and leg medical protection sleeve

(76) Inventor: John McTamney, Devon, PA (US)

Correspondence Address: EUGENE E. RENZ, JR., P.C., P.O. Box 2056, 205 North Monroe Street, Media, PA, 19063-9056, US

(21) Appl. No.: 09/753,526

(22) Filed: Jan. 3, 2001

Related U.S. Application Data
(60) Provisional application No. 60/174,294, filed on Jan. 3, 2000.

(51) Int. Cl. A61F 13/00

(52) U.S. Cl. 602/61; 602/60

(57) ABSTRACT

A sleeve-like medication application system is provided for body extremities such as an arm or leg where a medicament or lotion has been applied for therapeutic treatment. The sleeve is constructed of a material such as latex that conforms to body contours forming a tight covering over the medicated area preventing prescribed medicaments or ointments from oozing out, rubbing off or absorbed by clothing. Expansion bands at each end of the sleeve grip the arm or leg preventing the sleeve from creeping, rolling or becoming loose. The length and opening size of the sleeve may vary depending on the affected area and the physical makeup of the wearer.

### BACKGROUND OF THE INVENTION

Current skin care techniques of soothing or healing dry, itching or otherwise damaged skin are achieved by applying an appropriate medicated salve or lotion directly to an affected area. The lotion is absorbed by the skin, and in many cases, continual re-application is needed to heal the affected body area. Various devices have been proposed for dermatological treatment. In Palmer et al. BOOT WITH CONTINUOUS MEDICAMENT SUPPLY, U.S. Pat. No. 4,622,035 a boot is provided for the therapeutic or cosmetic treatment of the human foot. The boot has an impervious outer covering and includes a medicament supply network which provides a continuous supply of medicament to an absorbent lining around the inside of the outer covering. The medicament supply network contains a medicament reservoir located adjacent the opening of the boot and a series of supply fingers which extend along the inside of the contours of the outer covering to the extremities of the boot. A medicament of choice may be continuously supplied through the medicament supply network and transferred by the absorbent lining to the skin of the foot of the wearer.

In DeFina U.S. Pat. No. 5,614,202 a moisterizing glove is disclosed in which a middle layer saturated with lotion, an exterior layer of non-porous material formed to the top side of the middle layer, and an inner layer having a plurality of pores, formed to the bottom side of the middle layer, create a cavity for receiving and enveloping a human extremity, particularly a human hand.

Schonholtz U.S. Pat. No. 3,633,216 discloses a multi-layer, rubber surgical glove with at least one finger portion of double thickness to prevent punctures in the glove. Additionally, the multi-layer glove may contain an indicator substance, such as colored saline solution, inserted between the multi-layered areas. If a layer of the glove is ruptured, the indicator substance escapes and alerts the surgeon to discard and replace the ruptured glove.

The invention disclosed by Lengyel et al. U.S. Pat. No. 4,671,266 relates generally to protective coverings for wounds, blisters and the like, or as a protective covering in areas of the skin where blisters tend to form, to prevent blisters from forming. This invention relates especially to a blister bandage that has a high oxygen permeability, but is a liquid barrier and a bacteria barrier. The bandage compromises an aerated latex micro-sized hydro-entangled fabric having an adhesive disposed on at least one surface of the fabric. The properties of this fabric allow the fabric to remain soft, air permeable, flexible and strong. The fabric structure also makes the fabric conducive to providing a hydrophobic bacterial barrier thus lending itself for use as a blister bandage.

### SUMMARY OF THE INVENTION

Disclosures in the prior art tend to deal with devices for providing therapeutic treatment to specific body extremities, such as a boot or glove for the foot or hand, or a covering for a localized wound or blister. Prior inventions provide internal reservoirs of medicaments that are periodically dispensed to the affected area by the user. When medicaments are exhausted, the device is no longer available for reuse. Although traditional skin care techniques and pre-lubricated gloves are useful in many applications, they do not address a variety of other skin care problems, such as psoriasis. Also, skin care techniques utilize exposed, messy lotions that must be periodically re-applied.

Accordingly, it is an object of the present invention to provide a sleeve system that may be applied to an arm or leg over medication.

It is a further object of the present invention to provide a medication application sleeve system that may be washed and reused.

Another object of the present invention is to provide a medication application sleeve system that prevents prescribed creams, ointments, or medicaments from being rubbed off or absorbed by clothing.

It is still another object to provide skin care techniques and implements that avoid exposed, messy lotions.

A sleeve, marketed under the name DERMA-SLEEVE, is provided for body extremities such as an arm or leg where a medicament or lotion has been applied for therapeutic treatment. The sleeve, according to the principles of the present invention, is constructed of a material, such as latex, that conforms to the contours of the body extremity, forming a tight covering over the medicated area to prevent prescribed creams or ointments from oozing out, being rubbed off or absorbed by clothing. Expansion bands at each end of the sleeve grip the arm or leg, preventing the sleeve from creeping, rolling or becoming loose.

In accordance with one embodiment of the invention, the central portion of the sleeve is of a greater diameter than the expansion bands at opposite terminal ends so that the sleeve is spaced from the skin of the user about its periphery and likewise spaced from the medicament applied, for example, to the elbow area. This provides a blousing effect and aids in preventing the medication from being rubbed off or absorbed so that the full therapeutic effect of the medication can be realized. It is also believed that it minimizes irritation in the tender areas where the medication is applied and opens the pores for better absorption of the medication.

The longitudinal ridges forming the expansion band at opposite ends of the sleeve also prevent roll down of the sleeve and thus insures full containment of the medication and presents a more pleasing and less unsightly sleeve configuration.

In accordance with another embodiment of the present invention, the sleeve is of a frusto-conical shape to provide a better fit, for example, when the sleeve is used on the leg to cover the knee area and the expansion bands are located on the thigh and lower leg portion which are usually of a different size or diameter.

In accordance with still another embodiment of the present invention, the sleeve is provided with an enlarged pocket midway between opposing opposite ends to define a larger space between the sleeve and elbow and thus better circulation of air. It has been found that this arrangement promotes better retention of medicament in the knee and elbow area even when the knee and elbow points bend during normal movements of the arm and leg.

### BRIEF DESCRIPTION OF THE DRAWINGS

These and other objects of the present invention and various features and details of the operation and construction thereof are hereinafter more fully set forth with reference to the accompanying drawings, wherein:

FIG. 1 is a pictorial frontal view of a wearer of the derma-sleeve of the present invention shown slipped over an arm and leg.

FIG. 2 is a plan view of an arm sleeve showing the sleeve body and end treatments.

FIG. 3 is a plan view of a leg sleeve showing the sleeve body and end treatments.

FIG. 4 is a side elevational view of another embodiment of sleeve in accordance with the present invention which is of frusto-conical shape.

FIG. 5 is a view similar to FIG. 4, which is a modified form of this sleeve wherein the center section of the sleeve between the arm bands is of a greater diameter than the band portion, much like the embodiment shown in FIG. 2.

FIG. 6 is a side elevational view of still another embodiment of the sleeve in accordance with the present invention which incorporates an enlarged pocket to facilitate circulation of air in the elbow and knee region.

### DETAILED DESCRIPTION OF THE PREFERRED EMBODIMENT

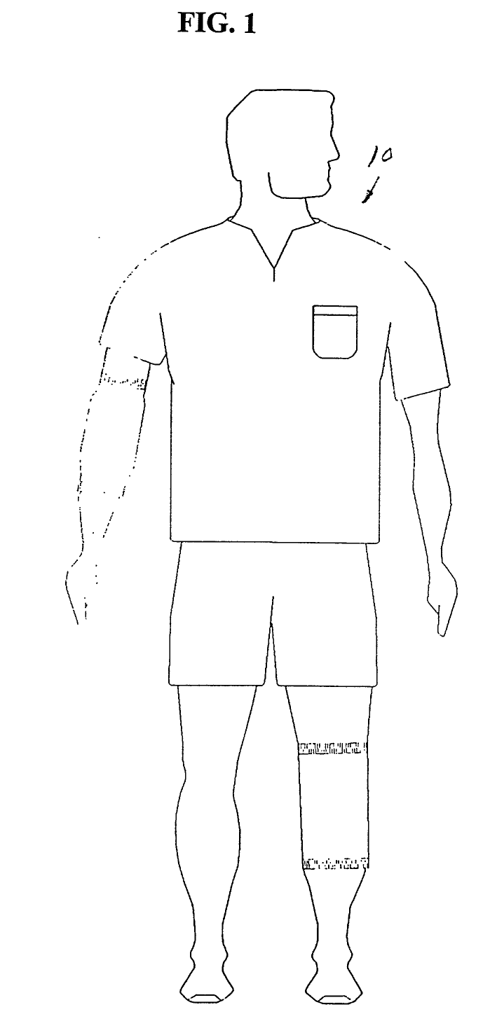

Referring now to the drawings and more particularly FIG. 1, a first embodiment of sleeve 20 of the invention is slipped over the arm of the wearer 10 covering the area treated with an ointment or cream. A second embodiment of sleeve 20 is shown on the leg of the wearer 10. In both cases, the length and opening size of the sleeve 20 may vary depending on the affected area and the physical makeup of the wearer.

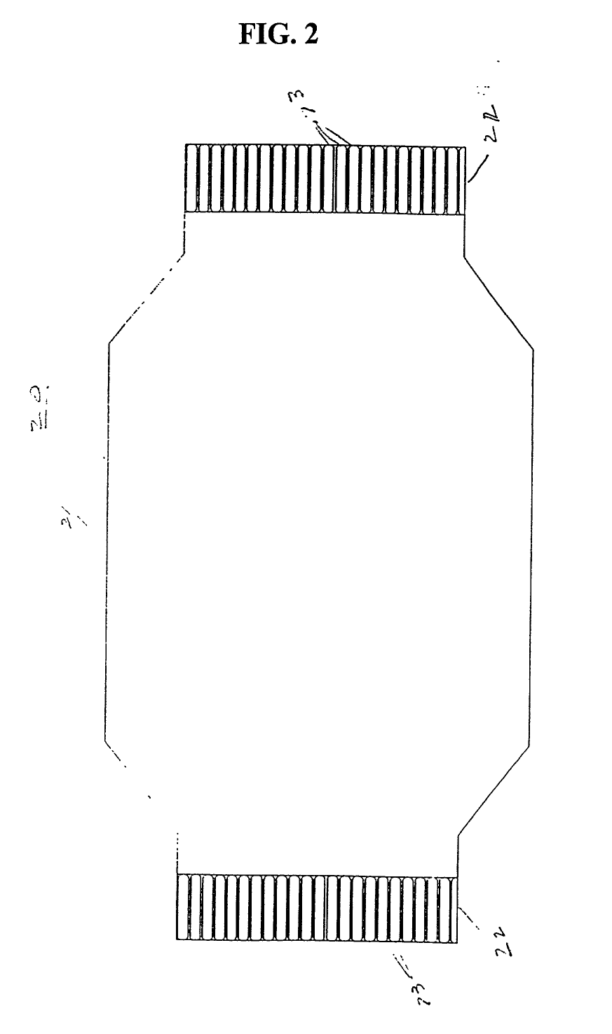

FIG. 2 illustrates a first embodiment of the sleeve 20 constructed to fit over a wearer's arm. Open at both ends a generally barrel shaped center portion 21 is comprised of a non-porous, stretchable material such as latex. Open ends 22 of sleeve 20 are provided with rigid elastic expansion bands 23 to tightly grip the arm thus containing the ointment or cream and preventing sleeve 20 from creeping, rolling or becoming loose.

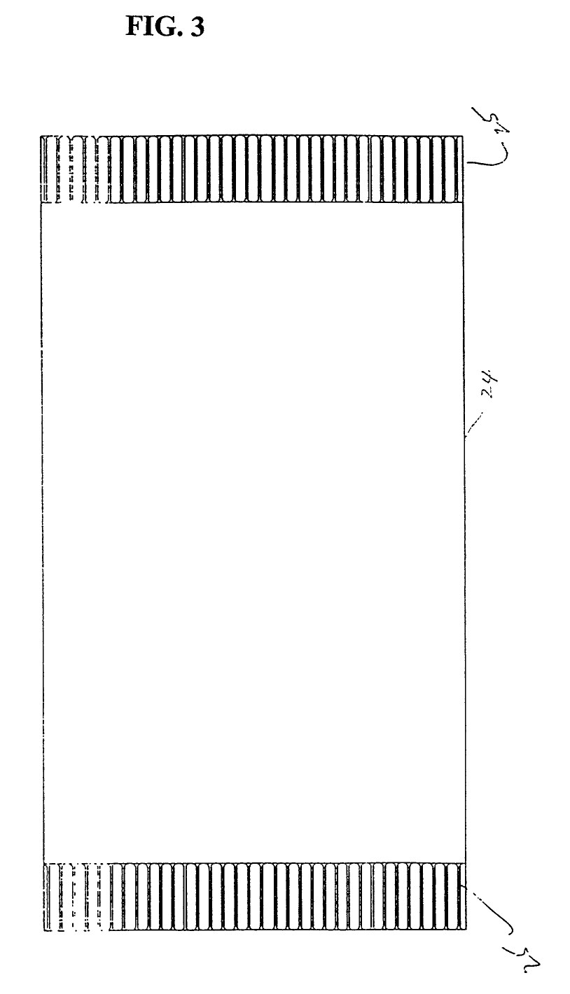

FIG. 3 illustrates a second embodiment of sleeve 20 constructed to fit over a wearer's leg. Open at both ends of sleeve 20 a generally cylindrical shaped center portion 24 is comprised of a non-porous, stretchable material such as latex. Open ends 25 of sleeve 20 are provided with ridged elastic expansion bands 26 to tightly grip the leg thus containing the ointment or cream and preventing sleeve 20 from creeping, rolling or becoming loose.

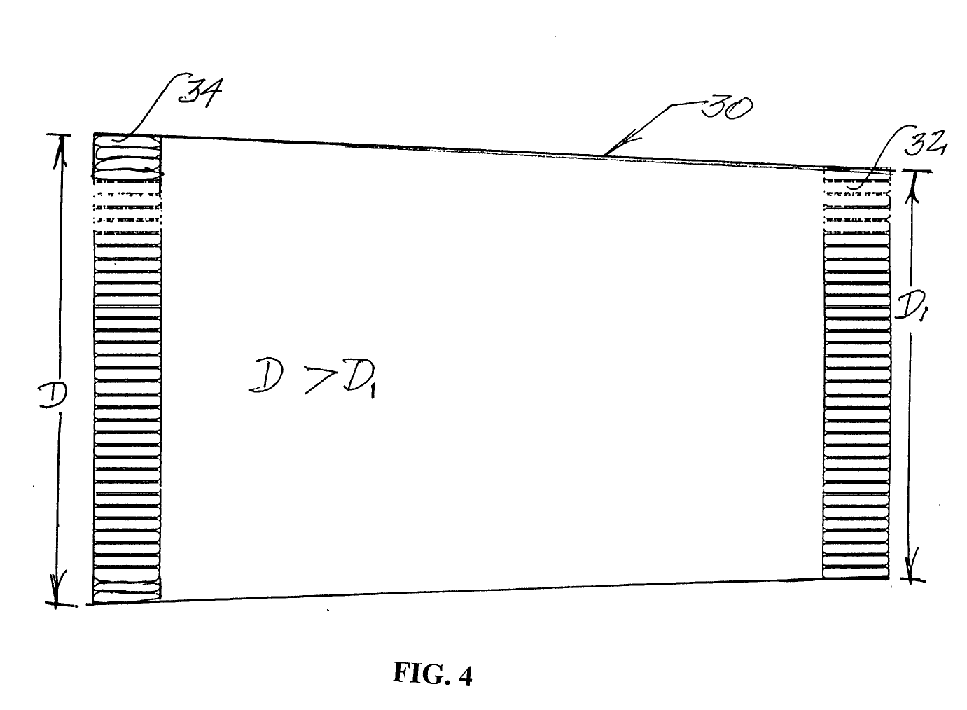

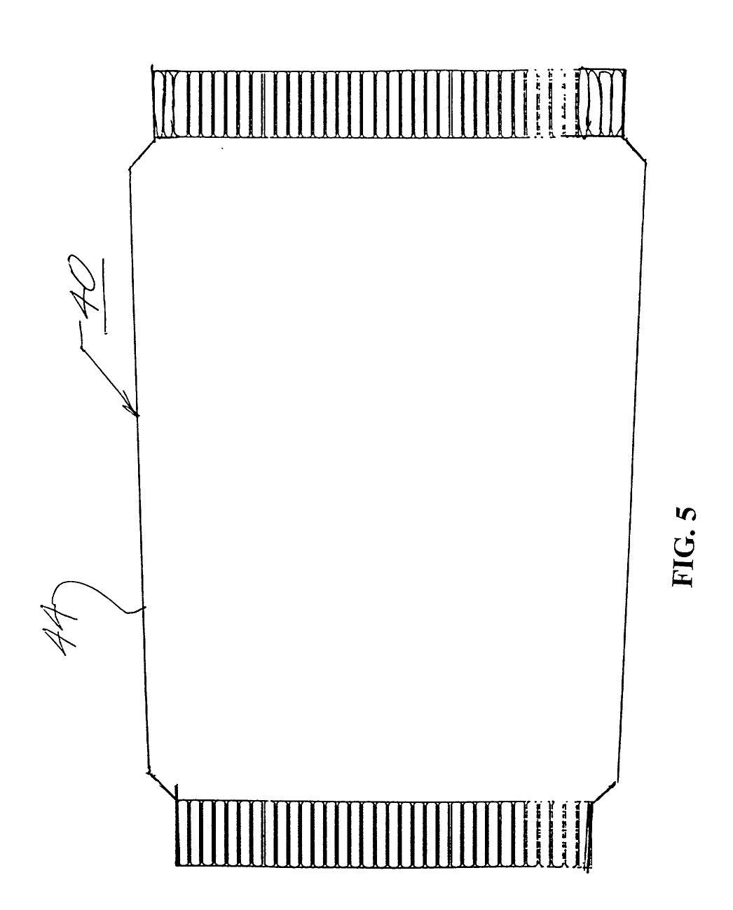

FIG. 4 shows another embodiment of sleeve constructed in accordance with the present invention. The sleeve which is generally designated by the numeral 30 in this instance is of elongated frusto-conical shape and has the longitudinal ribs 32 and 34 formed at opposite terminal ends for snugly engaging the arm or leg portion of the user. In this instance, the sleeve is adapted for use on the leg to cover the knee portion where typically the end of the sleeve circumscribing the thigh is of a greater diameter D than the portion surrounding the leg of the user below the knee D₁. The FIG. 5 embodiment is similar to that shown and is generally designated by the numeral 40 and has the enlarged central portion as indicated at 44 to provide the blousing effect as in the embodiment described above of FIG. 2.

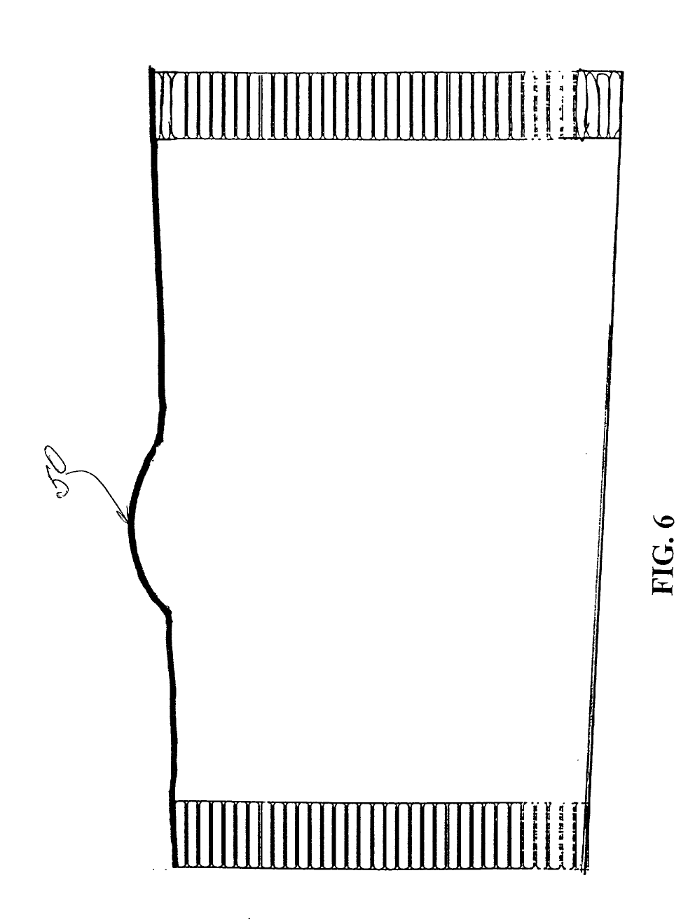

FIG. 6 shows still a further modification of a sleeve in accordance with the present invention. This sleeve may be a tubular arrangement as shown in FIG. 1 or the blousing arrangement described in connection FIG. 2. In this instance, the sleeve has a pocket 50 formed integrally therewith located approximately midway between the opposite terminal ends of the sleeve and which aligns with the elbow or knee of the user to permit better circulation of air in this region. This ensures retention of medication on the elbow or knee area even when there is normal bending of the elbow or knee.

Even though particular embodiments of the present invention have been illustrated and described herein, it is not intended to limit the invention and changes and modifications may be made therein within the scope of the following claims. For example, the sleeve may be made of a variety of flexible, elastic materials so that it is easy to apply. The materials may include neoprene, nitrile or latex. In general, the sleeve may be made of any materials which are non-absorbent and non-porous. The thickness of the film is preferably in the range of 0.004 to 0.25 mm and of a length anywhere between 8 inches to 20 inches.

Further, the sleeve of the present invention has useful application in the veterinary field and can be used for example in the leg, body and neck areas to prevent removal of medication or scratching of medicated areas covered by the sleeve.

What is claimed is:

1. A sleeve shaped and sized to cover body extremities where a medicament or lotion has been applied for therapeutic treatment, comprising:
a sleeve having an opening on both ends allowing a wearer to slip on to an arm or leg; constructed of materials that conform to body contours forming a tight covering over the medicated area; and having an integral covering section with expansion bands at each end to grip said arm or leg preventing said sleeve from creeping, rolling or becoming loose.

2. A sleeve as claimed in claim 1, wherein the center portion between the expansion bands at opposite terminal ends is of a greater diameter so that it is spaced from the skin of the user when it is applied to an arm or leg.

3. A sleeve as claimed in claim 1, wherein the sleeve is of frusto-conical shape and is particularly adapted for use on the leg of a user or the large end of the sleeve overlies the thigh and the small end of the sleeve engages the body below the knee.

4. A sleeve as claimed in claim 1, including means defining a pocket extending outwardly from the sleeve approximately midway between the opposite terminal ends of the sleeve.
